(19) United States
(12) Patent Application Publication
(10) Pub. No.: US 2026/0014002 A1
(43) Pub. Date: Jan. 15, 2026

(54) Breathable Prosthetic Liner

(71) Applicant: Alps South, LLC, St. Petersburg, FL (US)

(72) Inventors: Aldo Laghi, Pinellas Park, FL (US); David Carson, Clearwater, FL (US)

(21) Appl. No.: 19/263,040

(22) Filed: Jul. 8, 2025

Related U.S. Application Data
(60) Provisional application No. 63/669,793, filed on Jul. 11, 2024.

(51) Int. Cl. A61F 2/78; A61F 2/50

(52) U.S. Cl. CPC A61F 2/7812; A61F 2/5044; A61F 2002/5056; A61F 2002/7818

(57) ABSTRACT

A breathable prosthetic liner that includes an inner material in varying patterns that allows for air and fluid to move around the limb while maintaining a secure hold upon the amputee. The inner material is preferably made of nylon but may be a fleeced elastic and may contain a plurality of openings which may be along a central axis and offset.

### CROSS-REFERENCE TO RELATED APPLICATIONS

This application claims priority to U.S. Application No. 63/669,793, titled “Breathable Prosthetic Liner,” filed Jul. 11, 2024, which is hereby incorporated by reference in its entirety.

### FIELD OF THE INVENTION

The described invention relates to prosthetic liners. Specifically, the described invention relates to a prosthetic liner incorporating materials that create allows for breathability.

### DESCRIPTION OF THE BACKGROUND ART

Prosthetic liners have been in use since the 1970's, mostly custom made, and made of various materials. Prosthetic liners serve the purpose of providing comfort for an amputee when wearing a prosthetic limb as the liner allows for a cushion between the residual limb and the socket supporting the prosthetic limb. Silicone liners have been used since the 1980s in the prosthetic industry such as those described in U.S. Pat. No. 4,923,474 granted to Klasson and Kristinsson. Other examples of such liners include U.S. Pat. No. 5,728,168 to Laghi et al., U.S. Pat. No. 5,830,237 granted to Kania, U.S. Pat. No. 5,507,834 to Laghi et al., U.S. Pat. No. 5,443,525 to Laghi et al., and U.S. Pat. No. 5,728,168 to Laghi et al. Gel and urethane liners have also been used for prosthetic and orthotic purposes and, for the most part, have a fabric covering. The fabric covering is used to reinforce the underlying material (silicone, gel, urethane) and allows for extensibility to make it easier to don and doff the liner by rolling it on and off the residual limb

The issue with each of these prior liners is that they can sometimes be uncomfortable for a user due to lack of air flow thereby creating a lack of cooling. This is due to the direct skin contact between the inner gel/urethane layer which can be exacerbated when the user sweats due to heat or exercise. Current liner systems trap heat within the residual limb because it is enveloped by a liner and socket. Furthermore, the body's natural cooling system is sweat which gets trapped between the residual limb and liner interface making the issue worse. The buildup of sweat and heat can cause the liner to slip off the limb or leaves the amputee in a puddle at the bottom of the socket leading to skin issues and potential breakdown.

Other liner systems have been made that utilize holes to create airflow but these liners have had issues with the holes getting plugged up, not allowing enough air, and odor issues since the holes become difficult to clean. In addition, the holes cannot be too large otherwise vacuum will be affected thereby making it impossible for the user to keep their prosthetic attached and could cause hematomas to form on the user's leg due to fluid pooling in these voids. Furthermore, other liners can be stained by sweat buildup.

Therefore, it is an object of this invention to provide an improvement which overcomes the aforementioned inadequacies of the prior art devices and provides an improvement which is a significant contribution to the advancement of the breathable prosthetic liner art.

Another object of the invention is to provide a liner that allows for constant drainage of perspiration.

Another object of the invention is to provide a breathable liner that does not stain, has no bad odor, does not maintain soilage, and does not promote bacterial growth.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

The present invention relates generally to a breathable prosthetic liner that includes an inner material in varying patterns that allows for air and fluid to move around the limb while maintaining a secure hold upon the amputee. The inner material is preferably made of nylon but may also be a fleeced elastic and may contain a plurality of openings which may be along a central axis and offset.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a cross-section view of an embodiment of the present invention;

FIG. 2 is a cross-sectional view of an alternative embodiment of the present invention;

FIG. 3 is a perspective view of the interior layer of an alternative embodiment of the present invention;

FIG. 4 is a perspective view of the interior layer of an alternative embodiment of the present invention;

FIG. 5 is a perspective view of the interior layer of the embodiment shown in FIG. 2; and

FIG. 6 is a perspective view of the alternative embodiment shown in FIG. 3.

Similar reference numerals refer to similar parts throughout the several views of the drawings.

### DETAILED DESCRIPTION OF THE PREFERRED EMBODIMENT

The following description is of the best mode presently contemplated for carrying out the invention. This description is not to be taken in a limiting sense but is made merely for the purpose of describing one or more preferred embodiments of the invention. The scope of the invention should be determined with reference to the claims.

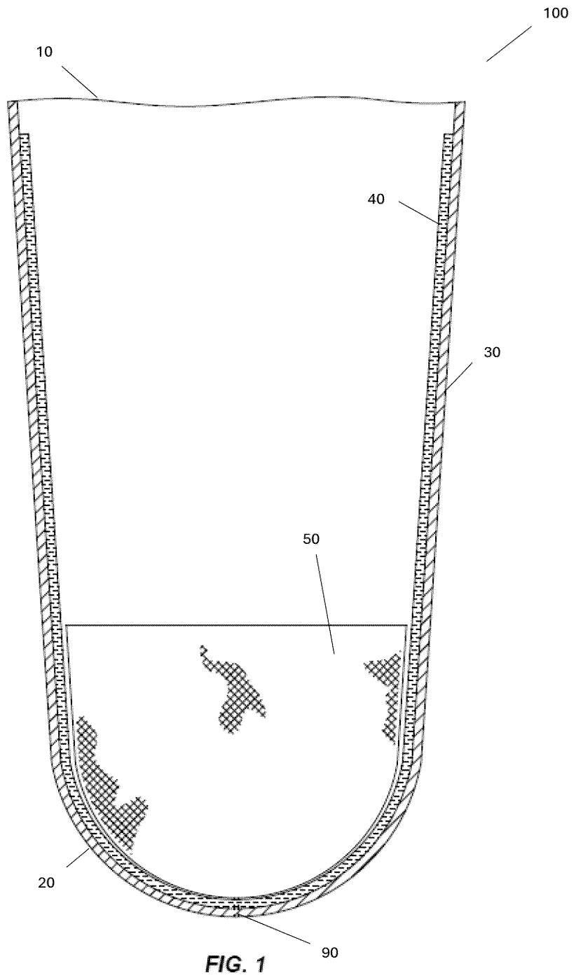

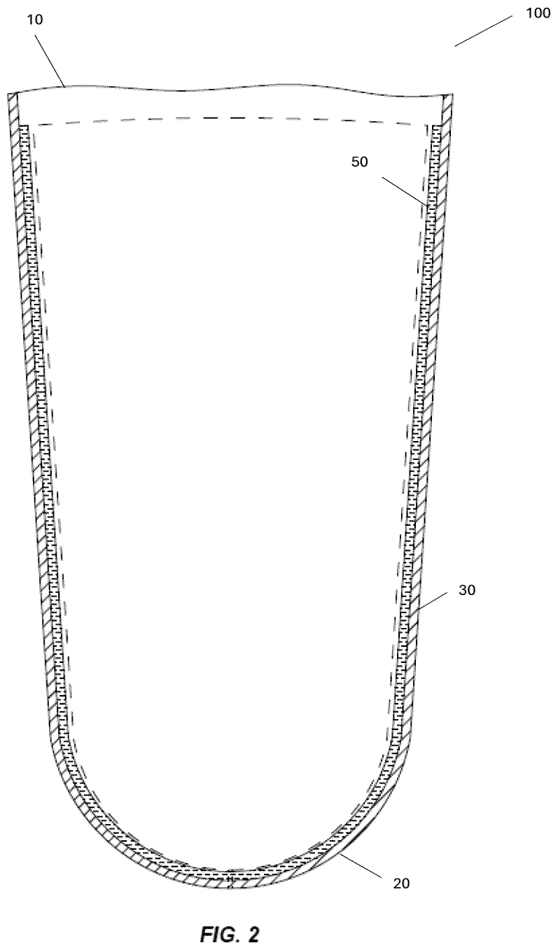

As shown in FIG. 1, the breathable liner 100 has an open proximal end 10, a closed distal end 20, an external fabric layer 30, and an interior thermoformable layer 40. Thermoformable layer 40 may be an elastomeric gel, urethane, or silicone. In one embodiment of the present invention, an inner material 50 is attached to the thermoformable layer 40 at the distal end 20. Alternatively, as shown in FIG. 2, the inner material 50 may extend the entire length of the breathable liner. In all embodiments, a through hole 90 which penetrates all layers of the breathable liner 100 is present at the distal end 20 that allows for air transmission. This through hole 90 allows for expulsion of air and uptake of cool air into the liner. During ambulation, the flexing of the socket and compression of layers acts as a pump that will expel hot humid air from the breathable liner 100 and draw in cool air from outside of the breathable liner 100 thereby cooling the limb and keeping moisture from building up.

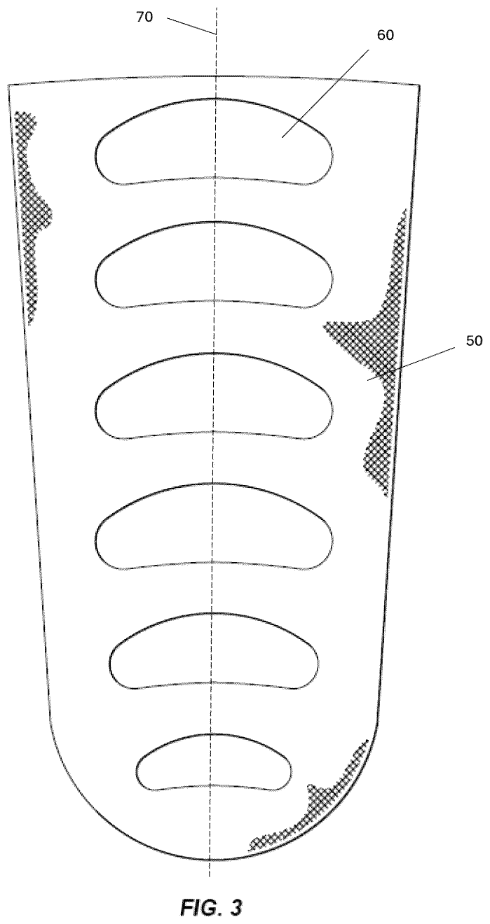

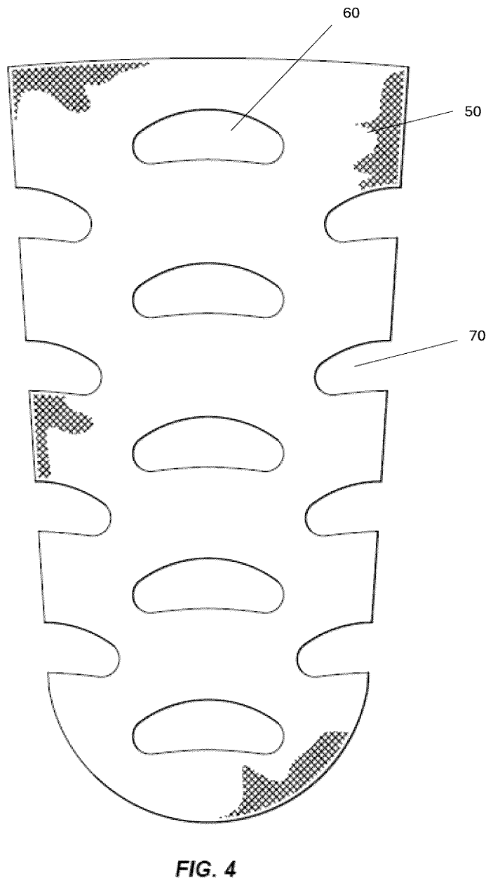

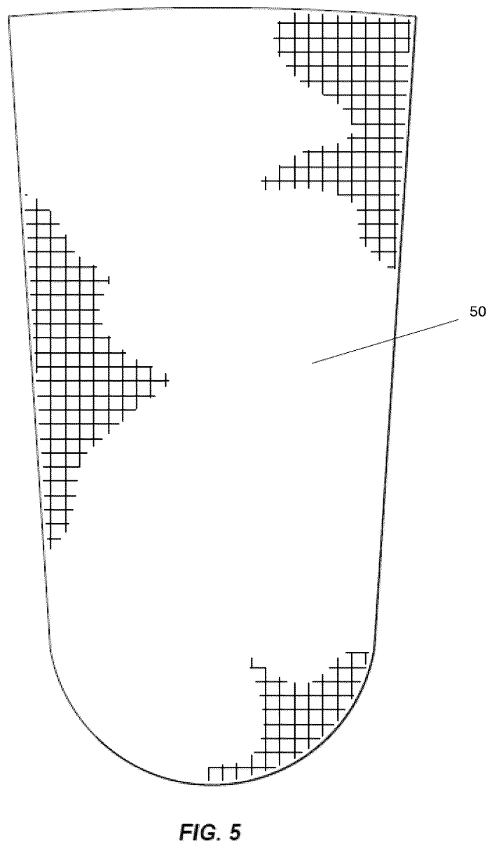

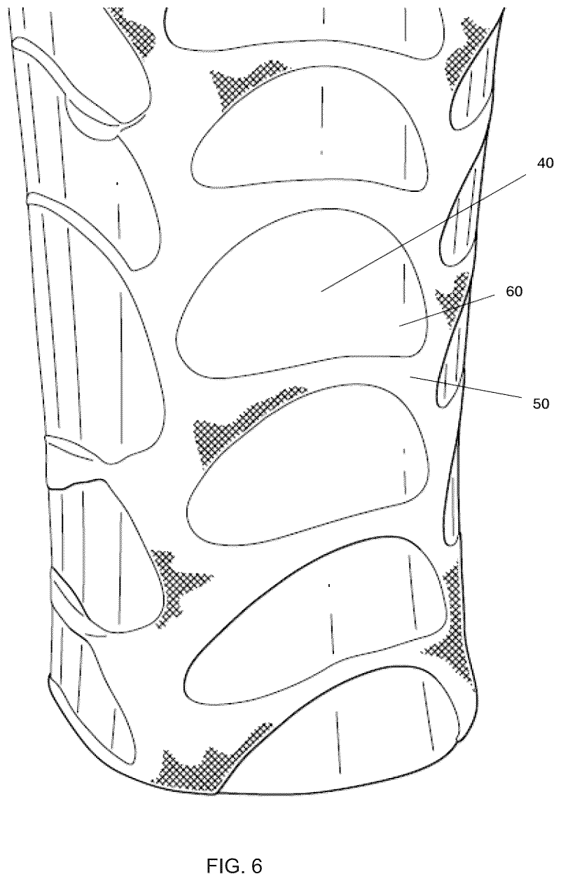

An alternative embodiment for the inner material 50 is shown in FIG. 3. In this version, the inner material 50 includes a plurality of central openings 60. These central openings 60, preferably ovular in shape and placed along a central axis 70, allow for the interior thermoformable layer 40 to bleed through and contact the skin when donned by a user. This can be seen in FIG. 6. Alternative embodiments of the inner material 50 are shown in FIGS. 4, 5, and 6. In FIG. 4, the inner material 50 includes side openings 80 in addition to central openings 60. Preferably, these side openings 70 are offset from central openings 60 to increase grip strength of the interior thermoformable layer 40. Finally, as shown in FIG. 5, the inner material 50 may simply be a very sheer material similar to a mesh. Preferably the inner material 50 in each of these embodiments is made from nylon but may be a fleeced elastic. In addition, the inner material 50 may be lined with silver to limit bacterial growth.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. A breathable prosthetic liner, comprising:
an open proximal end;
a closed distal end;
an external fabric layer;
an interior thermoformable layer;
an inner material incorporated within the thermoformable layer; and
a through hole at the distal end penetrating all layers of the breathable prosthetic liner, wherein the inner material allows for air and fluid movement while maintaining a secure fit on an amputee's residual limb.

2. The breathable prosthetic liner of claim 1, wherein the inner material extends the entire length of the breathable prosthetic liner.

3. The breathable prosthetic liner of claim 1, wherein the inner material comprises a plurality of central openings.

4. The breathable prosthetic liner of claim 3, wherein the central openings are ovular in shape and placed along a central axis.

5. The breathable prosthetic liner of claim 3, wherein the inner material further comprises side openings offset from the central openings.

6. The breathable prosthetic liner of claim 1, wherein the inner material is made from nylon or a fleeced elastic.

7. The breathable prosthetic liner of claim 6, wherein the inner material is lined with silver to limit bacterial growth.

8. A prosthetic liner system, comprising:
a tubular body having an open end and a closed end;
an outer fabric layer;
an inner elastomeric layer;
a breathable inner material layer attached to the inner elastomeric layer; and
a distal ventilation aperture extending through all layers of the tubular body.

9. The prosthetic liner system of claim 8, wherein the breathable inner material layer comprises a plurality of openings allowing for air and fluid movement while maintaining a secure fit on an amputee's residual limb.

10. The prosthetic liner system of claim 9, wherein the plurality of openings includes central openings arranged along a longitudinal axis of the tubular body.

11. The prosthetic liner system of claim 10, wherein the plurality of openings further includes side openings offset from the central openings.

12. The prosthetic liner system of claim 8, wherein the breathable inner material layer is made from nylon or a fleeced elastic material.

13. The prosthetic liner system of claim 12, wherein the breathable inner material layer is treated with an antimicrobial agent to limit bacterial growth.

14. The prosthetic liner system of claim 8, wherein the inner elastomeric layer is made from a material selected from the group consisting of silicone, gel, and urethane.

15. A method of manufacturing a breathable prosthetic liner, comprising:
forming a tubular structure with an open proximal end and a closed distal end;
applying an external fabric layer to the tubular structure;
applying an interior thermoformable layer within the tubular structure;
attaching an inner material to the interior thermoformable layer; and
creating a through hole at the distal end that penetrates all layers of the tubular structure.

16. The method of claim 15, wherein attaching the inner material to the interior thermoformable layer comprises attaching the inner material along the entire length of the tubular structure.

17. The method of claim 15, further comprising forming a plurality of openings in the inner material.

18. The method of claim 17, wherein forming the plurality of openings comprises creating central openings along a longitudinal axis of the tubular structure and side openings offset from the central openings.

19. The method of claim 15, wherein the inner material is made from nylon or a fleeced elastic material.

20. The method of claim 19, further comprising treating the inner material with an antimicrobial agent to limit bacterial growth.
